(19) United States
(12) Patent Application Publication
(10) Pub. No.: US 2002/0044913 A1
(43) Pub. Date: Apr. 18, 2002

(54) Cosmetics to support skin metabolism

(76) Inventor: Nathan D. Hamilton, Palo Alto, CA (US)

Correspondence Address: Barbara J. Luther, Chartered, 18124 Wedge Parkway, #516, Reno, NV, 89511, US

(21) Appl. No.: 09/782,652

(22) Filed: Feb. 12, 2001

Related U.S. Application Data
(60) Provisional application No. 60/181,793, filed on Feb. 11, 2000.
(60) Provisional application No. 60/223,584, filed on Aug. 7, 2000.

(51) Int. Cl. A61K 7/42; A61K 38/43

(52) U.S. Cl. 424/59; 424/94.1; 514/386

(57) ABSTRACT

Disclosed herein are cosmetic compositions to support skin metabolism and ameliorate the appearance of aging. Cosmetics such as masks, sunscreens, and lotions are particularly preferred. The compositions contain 0.01-30% of an antioxidant (preferably R-α-lipoic acid), 0.01-30% of a carnitine (preferably ALC), and optionally 0.01-15% of a coenzyme Q (preferably Q10), and/or 0.01-30% of a creatine. Also disclosed are methods of treatment of aged, photoaged, dry, lined or wrinkled skin. A method of protecting skin from the deleterious effects of sun exposure includes applying a sunscreen composition containing 0.001-10% antioxidant, 0.001-10% carnitine, and optionally 0.001-10% coenzyme Q and/or 0.140% creatine. The antioxidant, a carnitine, and optionally coenzyme Q and/or creatine fight age-related declines in skin mitochondrial function, which result in the appearance of aging.

This application claims the benefit of U.S. Provisional Application No. 60/181,793, filed Feb. 11, 2000, and U.S. Provisional Application No. 60/223,584, filed Aug. 7, 2000.

### TECHNICAL FIELD

The present invention is generally directed to cosmetics. More specifically, the present invention relates to the addition of the combination of lipoic acid, carnitine, and optionally coenzyme Q and/or creatine to cosmetic compositions.

### BACKGROUND OF THE INVENTION

The skin is the largest organ in the body. The skin protects the deeper tissues from injury, drying, and invasion by foreign organisms. The skin contains the peripheral endings of many sensory nerves, as well as blood vessels. It plays an important part in regulating body temperature and also has limited excretory and absorbing powers.

Skin consists principally of a layer of dense connective tissue, or dermis, and an external covering of epithelium, or epidermis. The epidermis is non-vascular and consists of stratified epithelium. It varies in thickness in different parts of the body. For example, the epidermis of the palms of the hands and the soles of the feet is thick, hard, and horny. The epidermis is divided into several layers. The deepest layer is the stratum basale. This layer contains dividing cells, which gradually replace those on the surface lost by abrasion. Next up is the stratum spinosum where the new cells develop internal fibrils and become connected to their neighbors. Overlying the stratum spinosum is the stratum granulosum, in which the skin cells have flattened and accumulate a precursor of keratin. The surface layer is the stratum corneum composed of scales or squamous plates, the remains of the cells and contain keratin, a fibrous protein.

The dermis is tough, flexible, and elastic. Like the epidermis, the dermis thickness varies, being thicker in the palms and soles, thicker on the back than the front of the chest, thicker on the outer surface of the limbs than the inner surface. The dermis consists of felted connective tissue with a varying amount of elastic fibers, numerous blood vessels, lymphatic vessels, nerves, smooth muscles, sweat glands, sebaceous glands, hair roots, fat cells, and papillae. Each papilla consists of very small and closely interlacing bundles of fine fibrous tissue with a few elastic fibers. Each contains a capillary loop; some contain tactile corpuscles for feeling.

Another factor, in addition to skin thickness, imparts the barrier function to the skin. Lipid layers in the skin form a waterproof barrier that prevents water loss from the skin. Water loss also contributes to the appearance of aged, dry or wrinkled skin. These lipids are predominantly ceramides, cholesterol, and fatty acids. In normal skin, if the barrier function is perturbed, the epidermis re-synthesizes the deficient lipids. Under certain conditions, however, a reduced capacity for re-synthesis may occur. This is especially so with aging or dry skin, where skin lipid levels are sub-normal.

The skin is the first line of defense of the immune system and shows the most visible signs of aging (West, Arch Dermatol 130(1): 87-95, 1994). As skin ages, it thins, develops wrinkles, discolors, and heals poorly. In youth, skin cells divide quickly in response to stress and trauma; but, over time, there are fewer and fewer actively dividing skin cells. Compounding the loss of regenerative capacity in aging skin is a concomitant loss of support tissues. The number of blood vessels in the skin decreases with age, reducing the nutrients that reach the skin. Also, aged immune cells less effectively fight infection. Nerve cells have fewer branches, slowing the response to pain and increasing the chance of trauma. In aged skin, there are also fewer fat cells, increasing susceptibility to cold and temperature changes. Old skin cells respond more slowly and less accurately to external signals. They produce less vitamin D, collagen, and elastin, allowing the extracellular matrix to deteriorate. As skin thins and loses pigment with age, more ultraviolet light penetrates and damages skin. To repair the increasing ultraviolet damage, skin cells need to divide to replace damaged cells, but aged skin cells are less capable of dividing (Fossel, REVERSING HUMAN AGING. William Morrow & Company, New York City, 1996).

Recent research has suggested that taking sufficient quantities of certain substances rejuvenates aged mitochondria, the failing powerhouses of cell metabolism. Numerous lines of evidence suggest that the organelles of cellular respiration, the mitochondria, degenerate with cellular aging (Shigenaga et al., PNAS 91: 10771, 1994). Unfortunately, the study of mitochondrial aging has been hampered because mitochondria isolated from older cells and host animals are fragile and heterogeneous. Hence the interpretation of any results is suspect as about half the mitochondria lyse during isolation. Recently a new method was developed for studying mitochondria in hepatocytes from old animals that avoids this problem (Hagen et al., PNAS 94: 3064-3069, 1997). Mitochondria from older animals are not only more fragile, but have about half the level of cardiolipin, a key lipid unique to mitochondria, without which they can not maintain sufficient membrane potential to control influx and exit of substances, which further impairs mitochondrial function. Furthermore, Hagen et al. show that in hepatocytes from older animals, the mitochondria have lower membrane potential and leak more toxic oxidants.

Another deterioration in skin is stasis dermatitis, which develops in the legs when venous circulation is impaired and may proceed to ulcers. The skin does not receive normal nutrition and is at risk for infection and contact dermatitis. Currently, stasis dermatitis is treated with emollients and/or topical steroids and control of edema.

Currently, to protect skin, cosmetics are applied to moisturize the skin, to balance the oil level of the skin and to provide protection against the adverse effects of sunlight, wind, and the harsh environment. Also cosmetics can be applied to the face and other parts of the body to even skin tone and texture and to hide pores, imperfections, fine lines and the like. Make-up compositions are generally available in the form of liquid or cream suspensions, emulsions, gels, pressed powders or anhydrous oil and wax compositions.

Historically, topical preparations have been complex mixtures. The compositions of this invention may contain other ingredients conventionally used in the art of skin care compositions, including but not limited to preservatives, preservative enhancers, and active ingredients in addition to the inventive combination.

The following patents feature cosmetic compositions containing lipoic acid: U.S. Pat. Nos. 5,089,269 (Cosmetic containing fine soft microcapsules, issued Feb. 18, 1992, to Shiseido Company Ltd.), 5,665,364 (Compositions for topical delivery of active ingredients, issued Sep. 9, 1997, to Procter & Gamble Co., 0.001-20% lipoic acid), 5,709,868 (Lipoic acid in topical compositions, issued Jan. 20, 1998, 0.25-5% lipoic acid derivative), 5,472,698 (Compositions for enhancing lipid production in skin, issued Dec. 5, 1995, to Elizabeth Arden Co., 0.0001-50% lipoic acid), 5,821,237 (Compositions for visually improving skin, issued Oct. 13, 1998, to Procter & Gamble Co., 0.1%-5% lipoic acid), 5,411,991 (Method of reducing hair growth employing sulfhydryl active compounds, issued May 2, 1995, 1-20% lipoic acid), and 5,607,980 (Topical compositions having improved skin feel, issued Mar. 4, 1997, to Procter & Gamble Co., 0.001-20% lipoic acid).

The following patents feature cosmetic compositions containing at least one carnitine: U.S. Pat. Nos. 5,843,476 (Slimming composition for topical treatment, issued Dec. 1, 1998, to L'Oreal), 5,741,816 (Hair-growth agent, issued Apr. 21, 1998, to Tanabe Seiyaku Co., Ltd.), 5,637,316 (Slimming composition for topical treatment, issued Jun. 10, 1997, to L'Oreal), 5,472,706 (Dry compositions for preparing submicron emulsions, issued Dec. 5, 1995, to Pharmos Corp., 40-90% carnitine in final dried emulsion), and 4,839,159 (Topical L-Camitine composition, issued Jun. 13, 1989, to Topicarn, Inc., 1-25% carnitine).

Coenzyme Q or ubiquinone has been used as a medicine or food supplement. For example, uses of ubiquinone include U.S. Pat. No. 6,090,414 (Method and composition to reduce cancer incidence); U.S. Pat. No. 6,086,190 (Food supplements); U.S. Pat. No. 6,080,788 (Composition for improvement of cellular nutrition and mitochondrial energetics); U.S. Pat. No. 6,080,388 (Cosmetic and dermatological sunscreen formulations); U.S. Pat. No. 6,063,432 (Fruit healthbar formulation); U.S. Pat. No. 6,048,846 (Compositions used in human treatment); U.S. Pat. No. 6,048,566 (Non-alcoholic beverage and process of making); etc.

Creatine has enjoyed increasing use as a nutritional additive by athletes. Other uses of creatine are discussed in U.S. Pat. No. 6,093,746 (Therapeutic agents for asthma); U.S. Pat. No. 6,071,962 (Oxa acids and related compounds for treating skin conditions); U.S. Pat. No. 6,060,512 (Method of using hydroxycarboxylic acids or related compounds for treating skin changes associated with intrinsic and extrinsic aging); U.S. Pat. No. 6,013,290 (Assemblage of nutrient beverages and regimen for enhancing convenience, instruction and compliance with exercise supplementation); U.S. Pat. No. 6,008,253 (Use of 3-guanidino propionic acid to increase endurance, stamina and exercise capacity); U.S. Pat. No. 6,008,252 (Method for increasing muscle mass); etc.

What are needed are improved cosmetics that are formulated to meet the true needs of older skin or avoiding the aging of skin. A survey of cosmetics Web sites uncovered no formula providing carnitine or lipoic acid. Such a formulation would also provide the latest in anti-aging compounds that have been shown to increase mitochondrial energy and stamina.

### SUMMARY OF THE INVENTION

It is an object of the present invention to improve cosmetics, preferably in formulations for skin with deficient mitochondrial metabolism. It is a further object to provide a combination of an effective amount of a suitable antioxidant and an effective amount of a carnitine in a wide variety of cosmetics.

A skin care composition in the form of a cream or lotion includes a cosmetically acceptable vehicle; and 1-30% of an antioxidant, 1-30% of a carnitine, and optionally 0.1-10% of a coenzyme Q and/or 1-30% of creatine, such that the cream or lotion delivers at least 40 mg/day of an antioxidant, 50 mg/day of a carnitine, and optionally at least 10 mg/day of coenzyme Q and/or at least 0.5 grams/day of creatine. Preferably the antioxidant is lipoic acid. More preferably, the antioxidant is R-α-lipoic acid. Preferably, the carnitine is ALC, and the coenzyme Q is coenzyme Q10.

Another embodiment is a cosmetic method of treating aged, photoaged, dry, lined or wrinkled skin. The method requires applying to the skin a cosmetic skin care composition containing 0.01-30% antioxidant, 0.01-30% carnitine, optionally 0.001-10% coenzyme Q and/or 0.1-40% creatine, and a cosmetically acceptable vehicle.

Another embodiment is a cosmetic method of improving the mitochondrial function in the skin. The method includes applying to the skin a cosmetic skin care composition containing 0.01-30% antioxidant, 0.01-30% carnitine, optionally 0.001-10% coenzyme Q and/or 0.1-40% creatine, and a cosmetically acceptable vehicle.

Another embodiment is a method of protecting skin from the deleterious effects of sun exposure. This method includes applying a sunscreen composition containing 0.001-10% antioxidant, 0.001-10% carnitine, and optionally 0.001-10% coenzyme Q and/or 0.1-40% creatine.

A preferred cosmetic of the present invention includes carnitine at such a concentration that when the cosmetic is applied, sufficient carnitine is administered. A preferred cosmetic of the present invention includes the antioxidant as R-α-lipoic acid at such a concentration that when the cosmetic is applied, sufficient antioxidant is administered.

### DETAILED DESCRIPTION

Currently available cosmetics lack a combination of two important ingredients: carnitine and lipoic acid. These two constituents are essential to discourage characteristics of aging by providing more energetic mitochondria. Recent research has shown precisely how these two compounds work to promote healthy mitochondria, which are the energy powerhouses of the cells. Mitochondria are responsible for the production of ATP and are present in relatively high numbers in essentially all cells of the body. The mitochondrial electron transport system consumes approximately 85% of the oxygen utilized by a cell. Cellular energy deficits caused by declines in mitochondrial function can impair normal cellular activities and compromise the cell's ability to adapt to various physiological stresses, a major factor in aging. Because of this high oxygen use, the mitochondria also have the highest production of harmful oxidants.

The inventive combination of carnitine, lipoic acid, and optionally coenzyme Q and/or creatine is provided in a skin product, preferably applied topically, that can slow the downward spiral that skin experiences with age. Such a product not only helps protect skin against the impairments of aging; it also may permit rejuvenated skin cells to restore youthful immune resistance and appearance. The combination can be used for both medical and cosmetic applications. It is important to note that although there are other available treatments for skin that address the loss of particular nutrients or proteins (such as moisturizers and products like Retin-A), the combination of camnitine, lipoic acid, and optionally coenzyme Q and/or creatine is needed to address the underlying cause of skin aging—mitochondrial senescence. Any optional ingredients must be compatible with lipoic acid and carnitine, such that the activity of the substance does not decrease unacceptably, preferably not to any significant extent, over a useful period (preferably at least about two years under normal storage conditions). For example, if strong oxidizing agents are incompatible with the inventive substance, such agents are avoided.

Oxidants damage mitochondria in three vital cell constituents. Oxidants damage DNA, lipids, and protein. The intra-mitochondrial DNA (mtDNA) have levels of oxidative damage which are at least 10-fold higher than those of nuclear DNA, which correlates with the 17-fold higher evolutionary mutation rate in mtDNA compared with nuclear DNA. mtDNA oxidation accumulates as a function of age, which has been shown in several species, including humans. This may lead to dysfunctional mitochondria. Mitochondrial protein damage is also age-related and may decrease energy production and increase oxidant production. Oxidative damage to mitochondrial lipids contributes to the decreasing fluidity of cell membranes with age. The lipid cardiolipin is a major component of the mitochondrial membrane and facilitates the activities of key mitochondrial inner membrane enzymes. The aged, damaged mitochondrial membrane cannot contain the oxidants nor can it maintain as high a polarity as the younger membrane.

Fatty acid oxidation is an important energy source for many tissues. The activity of carnitine-acetyl-carnitine exchange across the inner mitochondrial membrane is of great importance. The activity of this exchange reaction decreases significantly with age, which may be due to a lower intra-mitochondrial pool of carnitine. L-carnitine or acetyl-L-carnitine (ALC) has been shown to slow or reverse this age-related dysfunction. It also can reverse the age-related decrease in cardiolipin, age-associated decrease in mtDNA transcription, and decreased membrane potential. By itself, L-carnitine or ALC cannot correct the problem of excess oxidants. In fact, it was recently reported that carnitine supplementation increased oxidant production by 30% and decreased cell antioxidants markedly. Thus, ALC administration in older individuals may contribute to greater oxidative stress.

For the aged mitochondrial engines to run on all cylinders, both carnitine and lipoic acid are essential. Lipoic acid is an antioxidant. And R-α-lipoic acid is a mitochondrial enzyme that can help reverse the decline in metabolism seen with age. R-α-lipoic acid supplementation has been shown to 1) reverse the age-related decrease in oxygen consumption, 2) restore the age-related decline in mitochondrial membrane potential, 3) triple the ambulatory activity of aged rats, 4) significantly lower the age-related increase in oxidants, and 5) restore glutathione and ascorbic acid levels to youthful levels.

Clearly, both carnitine and lipoic acid contribute to restoration of age-related mitochondria function and metabolic activity in older people. This contributes to improvements in general skin health by supporting the epidermis and underlying structures, including immune system function, circulation, and muscles.

Carnitine is available in many forms, all of which are included in this invention. Carnitine and carnitine derivatives have been used as metabolites in animal husbandry and for human diet and therapy: U.S. Pat. No. 5,362,753 (Method of increasing the hatchability of eggs by feeding hens carnitine); U.S. Pat. No. 4,687,782 (Nutritional composition for enhancing skeletal muscle adaptation to exercise training); U.S. Pat. No. 5,030,458 (Method for preventing diet-induced carnitine deficiency in domesticated dogs and cats); U.S. Pat. No. 5,030,657 (L-carnitine supplemented catfish diet); U.S. Pat. No. 4,343,816 (Pharmaceutical composition comprising an acyl-carnitine, for treating peripheral vascular diseases); U.S. Pat. No. 5,560,928 (Nutritional and/or dietary composition and method of using the same); U.S. Pat. No. 5,504,072 (Enteral nutritional composition having balanced amino acid profile); U.S. Pat. No. 5,391,550 (Compositions of matter and methods for increasing intracellular ATP levels and physical performance levels and for increasing the rate of wound repair); U.S. Pat. No. 5,240,961 (Method of treating reduced insulin-like growth factor and bone loss associated with aging); etc. Most preferably, the carnitine is acetyl-L-carnitine.

A daily dosage of carnitine is about 5 mg to 8 g. Preferably the daily dose of carnitine is 25-1,000 mg. More preferably, the daily dose of carnitine is about 40-700 mg. Most preferably, the daily dose of carnitine is at least about 50 milligrams (0.05 g) per day.

By lipoic acid or thioctic acid is meant a mitochondrially active antioxidant which physiologically comprises a metabolically reactive thiol group. Mitochondrially active antioxidants including vitamins (especially C, E, B and D), glutathione, N-acetyl cysteine (NAC), lipoic acid, their derivatives, etc., have been used variously as human nutritional supplements and in dietary prophylaxis and therapy. For example, applications of lipoic acid have included U.S. Pat. No. 5,607,980 (Topical compositions having improved skin); U.S. Pat. No. 5,472,698 (Composition for enhancing lipid production in skin); U.S. Pat. No. 5,292,538 (Improved sustained energy and anabolic composition and method of making); U.S. Pat. No. 5,536,645 (Nutritive medium for the culture of microorganisms); U.S. Pat. No. 5,326,699 (Serum-free medium for culturing animal cells); etc. Preferably, the compound is at least one of glutathione, N-acetyl cysteine and lipoic acid. Most preferably, the compound is the R-enantiomeric form of lipoic acid. Metabolites of lipoic acid have been found to have a longer half life and also are suitable for supplementation.

A daily dosage of lipoic acid is about 5 mg to 8 g. Preferably the daily dose of lipoic acid is 10-1,000 mg. More preferably, the daily dose of lipoic acid is about 30-700 mg. Most preferably, the daily dose of lipoic acid is at least about 40 milligrams (0.04 g) per day.

Coenzyme Q (Q) or ubiquinone has been used as a medicine or food supplement. For example, uses of ubiquinone include U.S. Pat. No. 6,090,414 (Method and composition to reduce cancer incidence); U.S. Pat. No. 6,086,190 (Food supplements); U.S. Pat. No. 6,080,788 (Composition for Improvement of cellular nutrition and mitochondrial energetics);

U.S. Pat. No. 6,080,3 88 (Cosmetic and dermatological sunscreen formulations); U.S. Pat. No. 6,063,432 (Fruit healthbar formulation); U.S. Pat. No. 6,048,846 (compositions used in human treatment); U.S. Pat. No. 6,048,566 (Non-alcoholic beverage and process of making), etc.

Coenzyme Q, particularly Q10, is an important supplement. In groups of males and females ranging from 90-106 years, the prevalence of inadequate Q10 status was 40% for women and 24% for men. In women, the decreased Q10 was associated with impaired natural killer cell effectiveness (p<0.05), indicating decreased ability to fight infections and quickly eliminate individual cancer cells as they first develop. Q10 also appears to block programmed cell death, or apoptosis, through its action in the mitochondria (Kagan T et al, Ann NY Acad Sci 887:31-47, 1999). Furthermore, Q10 in its reduced from ofubiquinol-10 which is normally present in the blood, appears to protect human lymphocytes from oxidative damage to DNA (Tomasetti et al, Free Radic Biol Med 27 (9-10):1027-32, Nov. 1999). No important adverse effects have been reported from experiments using daily supplements of up to 200 mg Q10 for 6-12 months and 100 mg daily for up to 6 y. Overvad K et al. Eur J Clin Nutr 53(10):764-70, 1999.

Q10 also may contribute to anti-aging effect by protecting against atherosclerosis which also results from oxidative stress. Pedersen HS, et al. Biofactors 9(2-4): 319-23, 1999). Q10 also improves the tolerance of the senescent myocardium to aerobic and ischemic stress in human atrial tissue and rats. Q10 corrected the age-specific diminished recovery of function in older hearts so that older hearts recovered function at a similar rate to younger ones (Rosenfeldt FL et al. Biofactors 9(2-4): 291-9, 1999).

As for the supplemental dose of Q10, older Finnish men obtained benefit from 100 mg/day. A woman deficient in Q10 received 150 mg/kg and rapidly improved (Sobriera et al. Neurology 48:1283-43, 1997). Q10 has also been used at dose of about 200 mg/day to improve function in persons with hypertrophic cardiomyopathy. Based on this information, a supplemental dosage ranges from about 10 mg/day to about 500 mg/day. Preferably, the Q10 dose is about 100 mg/day.

Creatine has enjoyed increasing use as a nutritional additive by athletes. Other uses of creatine are discussed in U.S. Pat. No. 6,093,746 (Therapeutic agents for asthma); U.S. Pat. No. 6,071,962 (Oxa acids and related compounds for treating skin conditions); U.S. Pat. No. 6,060,512 (Method of using hydroxycarboxylic acids or related compounds for treating skin changes associated with intrinsic and extrinsic aging); U.S. Pat. No. 6,013,290 (Assemblage of nutrient beverages and regimen for enhancing convenience, instruction and compliance with exercise supplementation); U.S. Pat. No. 6,008,253 (Use of 3-guanidino propionic acid to increase endurance, stamina and exercise capacity); U.S. Pat. No. 6,008,252 (Method for increasing muscle mass); etc.

Because creatine intake is often decreased in older individuals, creatine supplementation is important to proper mitochondrial function. Many athletes have taken doses of creatine up to 75 grams a day for years without known adverse effects, aside from weight gain, often attributed to increased muscle mass. Creatine may be most beneficial when ingested with glucose, which increases creatine absorption. Often athletes ingest loading doses of 20 g/day divided into four doses for 5 days to one week. Then they take a maintenance dose of 5 g/day. Increased skeletal muscle strength and endurance has been seen in one week in older individuals (40-73) on a 20 g/day dose. It has been reported that 1.5 g-25 g/day were safe for period of at least a year. A suitable dosage range is 0.5 g/day to 50 g/day, preferably 1-10 grams per day and most preferably about 5 g/day. Creatine is available as a salt, monohydrate, phosphate and citrate.

In addition to the compositions mentioned above and the examples given below, the combination of carnitine, lipoic acid, and optionally coenzyme Q and/or creatine can be added to hair tonic, lotion and cream; shampoo; and conditioner. The inventive combination can be added to cleansers, normalizers, surface moisturizers, serums, anti-wrinkle creams, and long-lasting lipsticks and lip-glosses. The inventive combination also can be added to bulk powders or powder packets, for example, face masks and bath salts.

The inventive combination is also useful in a wide variety of new beauty products, including facial, neck, and hand patches; professional and home facial kits; chemical peels or post-peel gels and creams; products to lighten age spots and sunspots; products to minimize scars; products to use after laser treatment; moisturizing gloves and socks (NouveauDerm™, Self-Care, Emeryville, Cailf.); anti-small vein lotion (e.g., Veinish™ Swedish vein lotion, Self-Care); medicated creams; paraffin soaks; shave creams for men and women; depilatory products; anti-razor rash products (Tend Skin™, Self-Care); hair- and scalp-rejuvenating formulas.

The compositions of the subject invention may optionally comprise other active ingredients capable of functioning in different ways to enhance the benefits of the primary active substance and/or to provide other benefits. Examples of such substances include, but are not limited to, anti-inflammatory agents, antimicrobial agents, anti-androgens, sunscreens, sunblocks, chelators, depilation agents, desquamation agents, organic hydroxy acids, and natural extracts.

The compositions of the present invention may also include a natural extract of yeast, rice bran or the like such as are known in the art. Such extracts may enhance the skin appearance benefits of the present invention, and are preferably used in an amount of from 0.1% to about 20%, more preferably 0.5% to about 10%, also from 1% to about 5%. A natural extract of yeast is preferred.

The formulations and/or content of these products are on the product label or are otherwise publicly available.

Cosmetically Acceptable Vehicle

The composition according to the invention also comprises a cosmetically acceptable vehicle to act as a dilutant, dispersant or carrier for lipoic acid and carnitine in the composition, so as to facilitate its distribution when the composition is applied to the skin. Vehicles other than or in addition to water can include liquid or solid emollients, solvents, humectants, thickeners, and powders. An especially preferred nonaqueous carrier is a polydimethyl siloxane and/or a polydimethyl phenyl siloxane. Silicones may be those with viscosities ranging anywhere from about 10 to 10,000,000 mm²/s (centistokes) at 25° C. Especially desirable are mixtures of low and high viscosity silicones. These silicones are available from the General Electric Company under trademarks Vicasil, SE and SF and from the Dow Coming Company under the 200 and 550 Series. Amounts of silicone which can be utilized in the compositions of this invention range anywhere from 5% to 95%, preferably from 25% to 90% by weight of the composition.

The cosmetically acceptable vehicle usually forms from 5% to 99.9%, preferably from 25% to 80% by weight of the composition, and can, in the absence of other cosmetic ingredient!, form the balance of the composition. Penetration of the stratum comeum is essential for activity. Incorporation of lipoic acid and carnitine in such a liquid formulation greatly facilitates this penetration, as opposed to delivery from a powder. Preferably, the vehicle is at least 80% water, by weight of the vehicle. More preferably, the amount of water is at least 50% w/w of the inventive composition, and most preferably from 60 to 80% w/w.

Optional Skin Benefit Materials and Cosmetic Adjuncts

The inventive compositions preferably include sunscreens to lower the skin's exposure to harmful UV rays. Sunscreens include those materials commonly employed to block ultraviolet light. Illustrative compounds are the derivatives of PABA, cinnamate and derivatives of salicylate (other than ferulyl salicylate). For example, octyl methoxycinnamate and 2-hydroxy-4-methoxy benzophenone (also known as oxybenzone) can be used. Octyl methoxycinnamate and 2-hydroxy-4-methoxy benzophenone are commercially available under the trademarks, Parsol MCX and Benzophenone-3, respectively. The exact amount of sunscreen employed in the emulsions can vary, depending upon the degree of protection desired from the sun's UV radiation.

An oil or oily material may be present, together with an emollient to provide either a water-in-oil emulsion or an oil-in-water emulsion, depending largely on the average hydrophilic-lipophilic balance (HLB) of the emollient employed. Levels of such emollients may range from about 0.5% to about 50%, preferably between about 5% and 30% by weight of the total composition. Emollients may be classified under such general chemical categories as esters, fatty acids and alcohols, polyols and hydrocarbons.

Esters may be mono- or di-esters. Acceptable examples of fatty di-esters include dibutyl adipate, diethyl sebacate, diisopropyl dimerate, and dioctyl succinate. Acceptable branched chain fatty esters include 2-ethyl-hexyl myristate, isopropyl stearate and isostearyl palmitate. Acceptable tribasic acid esters include triisopropyl trilinoleate and trilauryl citrate. Acceptable straight chain fatty esters include lauryl palmitate, myristyl lactate, oleyl eurcate and stearyl oleate. Preferred esters include coco-caprylate/caprate (a blend of coco-caprylate and coco-caprate), propylene glycol myristyl ether acetate, diisopropyl adipate and cetyl octanoate.

Suitable fatty alcohols and acids include those compounds having from 10 to 20 carbon atoms. Especially preferred are such compounds such as cetyl, myristyl, palmitic and stearyl alcohols and acids.

Among the polyols which may serve as emollients are linear and branched chain alkyl polyhydroxyl compounds. For example, propylene glycol, sorbitol and glycerin are preferred. Also useful may be polymeric polyols such as polypropylene glycol and polyethylene glycol. Butylene and propylene glycol are also especially preferred as penetration enhancers.

Exemplary hydrocarbons that may serve as emollients are those having hydrocarbon chains anywhere from 12 to 30 carbon atoms. Specific examples include mineral oil, petroleum jelly, squalene and isoparaffins.

Another category of functional ingredients within the cosmetic compositions of the present invention are thickeners. A thickener will usually be present in amounts anywhere fronn 0.1 to 20% by weight, preferably from about 0.5% to 10% by weight of the composition. Exemplary thickeners are cross-linked polyacrylate materials available under the trademark Carbopol from the B. F. Goodrich Company. Gums may be employed, such as xanthan, carrageenan, gelatin, karaya, pectin and locust beans gum. Under certain circumstances, the thickening function may be accomplished by a material also serving as a silicone or emollient. For instance, silicone gums in excess of 10 centistokes and esters such as glycerol stearate have dual functionality.

Powders may be incorporated into the cosmetic composition of the invention. These powders include chalk, talc, kaolin, starch, smectite clays, chemically modified magnesium aluminum silicate, organically modified montmorillonite clay, hydrated aluminum silicate, fumed silica, aluminum starch octenyl succinate, and mixtures thereof.

Additional nutrients are important in older humans, including calcium, vitamin D, Vitamins B12, folic acid, B6, niacin, vitamins C or E, iron and zinc. Many of these nutrients have been found to be deficient in the diets of elders and can be appropriately supplemented in cosmetics, along with carnitine and thioctic acid.

Timed release agents are beneficial in cosmetics and cosmetic devices intended to be in contact with the skin for prolonged periods, particularly overnight. A preferred formulation provides lipoic acid and carnitine, optionally in combination with coenzyme Q10 and or creatine, in a timed release formulation to provide a steady supply of the nutrients to the mitochondria which work 24 hours a day. One method of accomplishing timed release is chemically combining the micronutrient(s) molecules with other molecules, which generally slows the process of making the micronutrient(s) available. Also the use of different salts of the micronutrients with different dissolution rates provides for gradual and appropriate release of the product.

Besides these methods, two other basic systems are used to control release: coating a core comprising the micronutrient(s) and excipients (coated system) and incorporating the micronutrient(s) into a matrix (matrix system). Coated systems involve the preparation of substance-loaded cores and coating the cores with release rate-retarding materials. Substance-loaded cores can be formulated as microspheres, granules, pellets or core tablets. There are many known core preparation methods, including, but not limited to, 1) producing granules by top spray fluidized bed granulation, or by solution/suspension/powdering layering by Wurster coating, 2) producing spherical granules or pellets by extrusion-spheronization, rotary processing, and melt pelletization; 3) producing core tablets by compression and coating with a release rate-retarding material; 4) producing microspheres by emulsification and spray-drying.

Matrix systems embed the micronutrient in a slowly disintegrating or non-disintegrating matrix. Rate of release is controlled by the erosion of the matrix and/or by the diffusion of the micronutrient(s) through the matrix. In general, the active substance, excipients and the release rate-retarding materials are mixed and then processed into matrix pellets. Matrix pellets can be formed by granulation, spheronization using cellulosic materials, or by melt pelletization using release retardant materials. An example of a cellulosic material is hydroxypropylmethyl-cellulose as the release rate-retarding material.

The rate of release can be further modified by blending coated or matrix pellets with different release rates of the same micronutrient to obtain the desired release profile. Pellets containing any of lipoic acid, carnitine, coenzyme Q10 or creatine can be blended to form a combination product.

Other adjunct minor components may also be incorporated into the cosmetic compositions. These ingredients may include coloring agents, opacifiers, and perfumes. Amounts of these other adjunct minor components may range anywhere from 0.001% up to 20% by weight of the composition. Water-insoluble pigments contribute to and are included in the total level of oil phase ingredients. Pigments suitable for use in the compositions of the present invention can be organic and/or inorganic. Also included within the term pigment are materials having a low color or luster such as matte finishing agents, and also light scattering agents. Examples of suitable pigments are iron oxides, acyglutamate iron oxides, ultramarine blue, D&C dyes, carmine, and mixtures thereof. Depending upon the type of composition, a mixture of pigments is normally used. The preferred pigments for use herein from the viewpoint of moisturization, skin feel, skin appearance and emulsion compatibility are treated pigments. The pigments can be treated with compounds such as amino acids, silicones, lecithin and ester oils.

The pH of the skin composition is preferably from about 4 to about 9, more preferably from about 6 to about 8.0. The water content of the compositions herein is generally from about 30% to about 98.89%, preferably from about 50% to about 95% and especially from about 60% to about 90% by weight.

Conventional cosmetic cream and lotion compositions as described, for example, in Sagarin, COSMETICS SCIENCE AND TECHNOLOGY, 2d Ed., Vol. 1, Wiley Interscience, 1972, and ENCYCLOPEDIA OF CHEMICAL TECHNOLOGY, 3d Ed., Vol. 7, John Wiley & Sons, 1984, are known to provide varying degrees of emolliency, barrier and water-retention (moisturizing) benefits.

Product Uses Form, and Packaging

In use, a small quantity of the composition (e.g., 1 to 100 ml) is applied to exposed areas of the skin, from a suitable container or applicator and, if necessary, it is then spread over and/or rubbed into the skin using the hand or fingers or a suitable device.

The cosmetic skin conditioning composition of the invention can be formulated as a lotion, a cream or a gel. The composition can be packaged in a suitable container to suit its viscosity and intended use by the consumer. For example, a lotion or cream can be packaged in a bottle or a roll-ball applicator, or a propellant-driven aerosol device or a container fitted with a pump suitable for finger operation. When the composition is a cream, it can simply be stored in a non-deformable bottle or squeeze container, such as a tube or a lidded jar. The composition may also be included in capsules such as those described in U.S. Pat. No. 5,063,507 (silicone-based anhydrous composition within a gelatin capsule), incorporated by reference herein. The invention accordingly also provides a closed container containing a cosmetically acceptable composition as herein defined.

Typically, dry ingredients are blended by mixing together, preferably shear mixing until homogeneous. With higher speed shear mixing, water based components are added gradually, and the batch is mixed for 35 minutes until dispersed. Oil-based ingredients are slowly added to the mixture with high shear mixing until dispersed. Silica can be added at this point and dispersed through the mixture. Next, there is prolonged mixing until there is no excess water on the surface. The mixture is then mixed for 15 minutes. The resulting make-up composition is ready for packing.

Convenient assays for the requisite bioactivities are described above or in the reference s cited herein. For example, cardiolipin content is readily assayed as referenced in Guan et al. Neurochem Int 25: 295-300, 1994; and oxidant production (DCFH) may be assayed as described by LeBel C P, Ischiropoulos H, and Bondy S C, Chem Res Toxicol 5:227-231, 1992. Assays for parameters of skin aging such as wrinkle density, skin thickness, density of age or sun spots, density of small veins (not varicose veins), skin tone, appearance and function, etc. are similarly well known in the art.

### Example 1

Facial masks, which contact the face for a prolonged period of time, are ideal cosmetics for the incorporation of carnitine, lipoic acid, and optionally coenzyme Q and/or creatine. There are a wide variety of mask formulas, containing for example, corn meal (to absorb dirt and oil), kaolin or bentonite clay (which tighten and purify the skin), natural honey (a humectant), aloe vera (a skin softener), jojoba and ginseng (relaxer), and apricot oil (for supple, smoother skin). An inventive mask additionally includes at least 0.1 grams of R-α-lipoic acid, at least 0.1 grams of L-carnitine, and optionally at least 10 mg Q10 and/or at least 0.5 grams creatine per mask.

### Examle 2

Hand creams also maintain prolonged contact with the skin and also are ideal for incorporation of carnitine, lipoic acid, and optionally coenzyme Q and/or creatine. The different brands and formulations are numerous. Common ingredients are well known in the art. One example is an herbal cream, which provides nutrients and moisture to the skin, by containing honey; vitamins A, E and C; as well as milk protein and squalene. We supplement the above formula with at least 0.1 grams of lipoic acid, at least 0.1 grams of carnitine, and optionally at least 10 mg Q10 and/or at least 0.5 grams creatine per application volume.

### Example 3

Sunscreens also maintain prolonged contact with the skin. Such a cosmetic incorporating carnitine, lipoic acid, and optionally coenzyme Q and/or creatine enhances the regeneration of cells and skin tissue, as well as fights premature aging. U.S. Pat. No. 6,019,992 issued Feb. 1, 2000, to Cheseborough-Pond discloses the following formulas from which 4-chromanone has been omitted. Sunscreens are applied to a much greater quantity of skin than the above hand cream, so the percentages of carnitine, lipoic acid, and optionally coenzyme Q and/or creatine are much lower. The compositions can be processed in conventional manner. They are suitable for cosmetic use. In particular the compositions are suitable for application to wrinkled, rough, flaky, aged and/or UV-damaged skin and/or dry skin and post-menopausal skin to improve the appearance and the feel thereof as well as for application to healthy skin to prevent or retard deterioration thereof.

| | |
|---|---|
| INGREDIENT | % w/w |

OIL-IN-WATER EMULSION

| | |
|---|---|
| DI Water | 73.40 |
| Carbomer | 0.30 |
| Disodium EDTA | 0.10 |
| Glycerin | 3.00 |
| Polysorbate 20 | 2.50 |
| Butylene Glycol | 2.00 |
| Methylparaben | 0.30 |
| Triethanolamine | 0.30 |
| Isopropyl Myristate | 4.00 |
| Octyl Palmitate | 3.00 |
| Cetyl Alcohol | 1.00 |
| Carnitine | 1.00 |
| Lipoic Acid | 1.00 |
| Creatine | 0.90 |
| Dimethicone, 100 cst | 0.50 |
| Beeswax | 0.30 |
| Propylparaben | 0.10 |
| Coenzyme Q10 | 0.10 |
| Germall II | 0.10 |
| Fragrance | 0.10 |
| Total | 100.00 |
| DI Water | 71.20 |
| Xanthan Gum | 0.20 |
| Disodium EDTA | 0.10 |
| Glycerin | 5.00 |
| Butylene Glycol | 2.00 |
| Methylparaben | 0.30 |
| 4-chromanone | 2.00 |
| Isopropyl Myristate | 4.00 |
| Octyl Palmitate | 3.00 |
| Cetyl Alcohol | 1.00 |
| Carnitine | 1.00 |
| Lipoic Acid | 1.00 |
| Creatine | 0.90 |
| Dimethicone, 100 cst | 0.50 |
| Steareth-2 | 0.40 |
| Steareth-21 | 3.00 |
| Coenzyme Q10 | 0.10 |
| Propylparaben | 0.10 |
| Germall II | 0.10 |
| Fragrance | 0.10 |
| Total | 100.00 |

WATER-IN-OIL EMULSION

| | |
|---|---|
| DI Water | 62.30 |
| Disodium EDTA | 0.10 |
| Glycerin | 3.00 |
| Propylene Glycol | 2.00 |
| Sodium Chloride | 0.70 |
| Methylparaben | 0.30 |
| Cyclomethicone | 14.00 |
| Isopropyl Myristate | 5.00 |
| Octyl Palmitate | 3.00 |
| Dimethicone Copolyol | 2.50 |
| Carnitine | 1.00 |
| Lipoic Acid | 1.00 |
| Creatine | 0.90 |
| Dimethicone, 100 cst | 0.50 |
| Coenzyme Q10 | 0.10 |
| Beeswax | 0.30 |
| Propylparaben | 0.10 |
| Germall II | 0.10 |
| Fragrance | 0.10 |
| Total | 100.00 |

HYDRO-GEL

| | |
|---|---|
| DI Water | 81.85 |
| Butylene Glycol | 5.00 |
| PPG-5-Ceteth 20 | 5.00 |
| Glycerin | 3.00 |
| Carboner | 1.20 |
| Triethanolamine 99% | 1.20 |
| Carnitine | 1.00 |
| Lipoic Acid | 1.00 |
| Creatine | 0.90 |
| Methylparaben | 0.30 |
| Polysorbate 20 | 0.25 |
| Coenzyme Q10 | 0.10 |
| Disodium EDTA | 0.10 |
| Germall II | 0.10 |
| Total | 100.00 |

ANHYDROUS SERUM

| | |
|---|---|
| Cyclomethicone | 71.40 |
| Isopropyl Myristate | 5.00 |
| Octyl Palmitate | 3.00 |
| Polyglycerol-6 | 5.00 |
| Dioleate | |
| Butylene Glycol | 4.00 |
| Dimethicone, 100 cst | 5.00 |
| Carnitine | 1.00 |
| Lipoic Acid | 1.00 |
| Creatine | 0.90 |
| Beeswax | 0.30 |
| Propylparaben | 0.20 |
| Coenzyme Q10 | 0.10 |
| Fragrance | 0.10 |
| Total | 100.00 |

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

We claim:

1. A skin care composition in the form of a cream or lotion comprising:
(a) a cosmetically acceptable vehicle; and
(b) 1-30% of an antioxidant and 1-30% of a carnitine; and
(c) optionally 0.01-10% of coenzyme Q and/or 1-30% of creatine;
whereby the cream or lotion delivers at least 40 mg/day of an antioxidant, 50 mg/day of a carnitine, and optionally at least 10 mg/day of coenzyme Q and/or at least 0.5 grams/day of creatine.

2. A skin care composition according to claim 1 wherein the antioxidant is lipoic acid.

3. A skin care composition according to claim 1 wherein the antioxidant is R-α-lipoic acid.

4. A skin care composition according to claim 1 wherein the carnitine is ALC.

5. A skin care composition according to claim 1 wherein the coenzyme Q is coenzyme Q10.

6. A cosmetic method of treating aged, photoaged, dry, lined or wrinkled skin, the method comprising applying to the skin a cosmetic skin care composition comprising:
(a) from 0.01-30% antioxidant and from 0.01-30% a carnitine;
(b) optionally from 0.001-10% coenzyme Q and/or from 0.1-40% creatine; and
(c) a cosmetically acceptable vehicle.

7. A cosmetic method of improving the mitochondrial function in the skin, the method comprising applying to the skin a cosmetic skin care composition comprising:
(a) from 0.01-30% antioxidant, from 0.01-30% a carnitine;
(b) optionally from 0.001-10% coenzyme Q and/or from 0.1-40% creatine; and
(c) a cosmetically acceptable vehicle.

8. A method of protecting skin from the deleterious effects of sun exposure, the method comprising applying a sunscreen composition containing 0.001-10% antioxidant, 0.001-10% carnitine, and optionally 0.001-10% coenzyme Q and/or 0.140% creatine.
